SEQUENCE LISTING (3 sequences)

SEQ ID NO: 1 - DNA, 2197 bp, Homo sapien
ccatcctaatacgactcactatagggctcgagcggccgcccgggcaggtgcggctgcgcgccttcctgctccgagtctctgcacctccctcaggagcctgtcagcctggccctcgtgagaggggcgccagcccagcagcctgctctggggcaccctcccctacctgaagggcacagggtttcgggagttttccaccatgactattgccctgctgggttttgccatattcttgctccattgtgcgacctgtgagaagcctctagaagggattctctcctcctctgcttggcacttcacacactcccattacaatgccaccatctatgaaaattcttctcccaagacctatgtggagagcttcgagaaaatgggcatctacctcgcggagccacggtgggcagtgaggtaccggatcatctctggggatgtggccaatgtatttaaaactgaggagtatgtggtgggcaacttctgcttcctaagaataaggacaaagagcagcaacacagctcttctgaacagagaggtgcgagacagctacaccctcatcatccaagccacagagaagaccttggagttggaagctttgacccgtgtggtggtccacatcctggaccagaatgacctgaagcctctcttctctccaccttcgtacagagtcaccatctctgaggacatgcccctgaagagccccatctgcaaggtgactgccacagatgctgatctaggccagaatgctgagttctattatgcctttaacacaaggtcagagatgtttgccatccatcccaccagcggtgtggtcactgtggctgggaagcttaacgtcacctggcgaggaaagcatgagctccaggtgctagctgtggaccgcatgcggaaaatctctgagggcaatgggtttggcagcctggctgcacttgtggttcatgtggagcctgccctcaggaagcccccagccattgcttcagtggtggtgactccaccagacagcaatgatggtaccacctatgccactgtactggtcgatgcaaatagctcaggagctgaagtggagtcagtggaagttgttggtggtgaccctggaaagcacttcaaagccatcaagtcttatgcccggagcaatgagttcagtttggtgtctgtcaaagacatcaactggatggagtaccttcatgggttcaacctcagcctccaggccaggagtgggagcggcccttatttttattcccagatcaggggctttcacctaccaccttccaaactgtcttccctcaaattcgagaaggctgtttacagagtgcagcttagtgagttttcccctcctggcagccgcgtggtgatggtgagagtcaccccagccttccccaacctgcagtatgttctaaagccatcttcagagaatgtaggatttaaacttaatgctcgaactgggttgatcaccaccacaaagctcatggacttccacgacagagcccactatcagctacacatcagaacctcaccgggccaggcctccaccgtggtggtcattgacattgtggactgcaacaaccatgcccccctcttcaacaggtcttcctatgatggtaccttggatgagaacatccctccaggcaccagtgttttggctgtgactgccactgaccgggatcatggggaaaatggatatgtcacctattccattgctggaccaaaagctttgccattttctattgacccttacctggggatcatctccacctccaaacccatggactatgaactcatgaaaagaatttataccttccgggtaagagcatcagactggggatccccttttcgccgggagaaggaagtgtccatttttcttcagctcaggaacttgaatgacaaccagcctatgtttgaagaagtcaactgtacagggtctatctgccaagactggccagtagggaaatcgataatgactatgtcagccatagatgtggatgagcttcagaacctaaaatacgagattgtatcaggcaatgaactagagtattttgatctaaatcatttctccggagtgatatccctcaaacgcccttttatcaatcttactgctggtcaacccaccagttattccctgaagattacagcctcagatggcaaaaactatgcctcacccacaactttgaatattactgtggtgaaggaccgcgggagaatcaggatc

SEQ ID NO: 2 - protein, 667 aa, Homo sapien
MTIALLGFAIFLLHCATCEKPLEGILSSSAWHFTHSHYNATIYENSSPKTYVESFEKMGIYLAEPRWAVRYRIISGDVANVFKTEEYVVGNFCFLRIRTKSSNTALLNREVRDSYTLIIQATEKTLELEALTRVVVHILDQNDLKPLFSPPSYRVTISEDMPLKSPICKVTATDADLGQNAEFYYAFNTRSEMFAIHPTSGVVTVAGKLNVTWRGKHELQVLAVDRMRKISEGNGFGSLAALVVHVEPALRKPPAIASVVVTPPDSNDGTTYATVLVDANSSGAEVESVEVVGGDPGKHFKAIKSYARSNEFSLVSVKDINWMEYLHGFNLSLQARSGSGPYFYSQIRGFHLPPSKLSSLKFEKAVYRVQLSEFSPPGSRVVMVRVTPAFPNLQYVLKPSSENVGFKLNARTGLITTTKLMDFHDRAHYQLHIRTSPGQASTVVVIDIVDCNNHAPLFNRSSYDGTLDENIPPGTSVLAVTATDRDHGENGYVTYSIAGPKALPFSIDPYLGIISTSKPMDYELMKRIYTFRVRASDWGSPFRREKEVSIFLQLRNLNDNQPMFEEVNCTGSICQDWPVGKSIMTMSAIDVDELQNLKYEIVSGNELEYFDLNHFSGVISLKRPFINLTAGQPTSYSLKITASDGKNYASPTTLNITVVKDRGRIRI

SEQ ID NO: 3 - DNA, 1455 bp, Homo sapien
atcatgatcataagacattccatgagtgagaaaacagggtgatacaatagaataggtcaattctccatacggaccagcatcaaaatccagagcatttatagagcatatggtagaggaaataggcagattttctggaacaatacagctgaagcttgagaacataaactgaggtgcatggtcgttatcatccaggacactgacaaacacaactgcaaaagaaaaatgtttcttttctgcatctgaagcttggacagttaaggtgaattttgtcattttttcataatccagaggtttaatcaaataaagaactccagtgttttcttctaagtaaaaatgtcccttctcatttccagagatgatgttgtagatgatttctgcatgtgaccctgtgtcacggtcatttgctgagaccacagtgatgtgactccctagaggggtgctttccttgacatgggtgtgatagctcaggctgctgaagtttgggggattgtcattgacatcaagtacttgtattgatatgacagctgtggaactcaatggagggcagccactgtcagatgccagaatgacaagctcatggctagcacttgcttctctgtccagactgtgaagcaacacaagataaccgacttgcttataaggatattctgaatgaaagaacttagtttccacatgaaaattgttctgtgaattaccactgatgatggaatattcaacatatgtgttttcacgggtccagtcatggtcgatgtttgaaaatgtaacaagcgtgcttccaaccagagcatcctcacttaggctaagattataggatttgactgtgaattcaggggcataattgttcatatcttctattcctatctccactaaagtaagagctctcaggtcaggatttccaccatcactggcttccacaagaaattgagttgttgatattgtatccagaagtaatacgggactgatagtaaatattgtgcctgaaaaataagacatagtatttcaacactgtgaggtgcagttttgatgatccatttaatcggcagttgactatccaatgtctactaatatgtgtttaattctttgttagatctgttaagtcacaatgaacaatgactccaaatagaaatacaaattattgtatttatatgtttataacatgtaagatacagaaataatattcatgacattcactctgtgaaatatgtgtgctgcttgaggttgctgttgccacaaattcctctgaattttgtttttgtatagtaagttgttccactattcttaaaaaatatttttgtttgtaaaatttataccccattctactacccacaaacacatgtataaagatattctggcataaagatacttattctggaaatacttgttgatattcaagccaaatgaaagcagtaaaaatgaaaaaaaaaaaaaaaaaaaaaaattgctgcggccgcaagg

(19) United States
(12) Patent Application Publication
(10) Pub. No.: US 2003/0023996 A1
(43) Pub. Date: Jan. 30, 2003

(54) HUMAN FAT-2 (HFAT2)

(76) Inventors: Filippo Randazzo, San Fracisco, CA (US); David Duhl, Oakland, CA (US); Gary J. Miller, Denver, CO (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/358,635

(22) Filed: Jul. 21, 1999

Related U.S. Application Data
(60) Provisional application No. 60/093,924, filed on Jul. 23, 1998.

(51) Int. Cl. A01K 67/00; C07H 21/04

(52) U.S. Cl. 800/13; 800/8; 536/23.1; 536/23.5; 435/1.1

(57) ABSTRACT

Human fat gene (hfat-2), and the amino acid sequence encoded by the hfat-2 gene are disclosed. hfat-2 is expressed at higher levels in normal prostate cells and in benign prostate cancer cell lines than in metastatic prostate cancer cell lines and tissue.

This application claims priority to Provisional Application Serial No. 60/093,924, filed Jul. 23, 1998.

### FIELD OF THE INVENTION

The invention relates to a human fat-2 (hfat-2) gene and polypeptide.

### BACKGROUND OF THE INVENTION

Cancer is a complex, multistep process involving the aberrant expression of genes that regulate cell growth, as described in Barba et al., Advances in Nutrition and Cancer (Zappia ed. Plenum Press N.Y. 1993), pp. 45-57. Tumor suppressor genes, the loss of function of which has been implicated in cancer, can be inactivated or lost by mutation, chromosome loss, mitotic recombination or gene conversion as described in Lasko et al., Annu. Rev. Genet. 25:281-314 (1991). The identification of tumor suppressor genes and other genes that play a role in cancer development is important to preventing, understanding, and treating cancer, and there is a need in the art for discovery of the role of such genes.

### SUMMARY OF THE INVENTION

This need is met in part by the identification of a gene designated “hfat-2” for human fat-2.

The invention relates to a hfat-2 polypeptide encoded by at least SEQ ID NOS: 1 and 3.

The invention also relates to a hfat-2 polypeptide of at least 50 consecutive amino acids of SEQ ID NO: 2 wherein the polypeptide exhibits 60% or more of native activity of the hfat-2 protein.

The invention further relates to a polynucleotide that encodes at least 50 consecutive amino acids of hfat-2 from SEQ ID NO: 2.

The invention also relates to a polynucleotide sequence encoding a biologically functional portion of a polypeptide sequence of SEQ ID NO: 2.

Still another embodiment of the invention is a polynucleotide sequence encoding SEQ ID NO: 2.

Another embodiment of the invention is a polynucleotide comprising the sequence of SEQ ID NO: 1.

Another embodiment is an mRNA molecule capable of hybridizing to a h-fat2 polynucleotide under stringent conditions.

Another embodiment is a polypeptide translated from an mRNA molecule capable of hybridizing to an h-fat2 polynucleotide under stringent conditions.

Another embodiment is a method of producing an h-fat2 polypeptide by introducing into a host cell a polynucleotide that encodes the polypeptide, and expressing the polypeptide in the host cell.

Another embodiment of the invention is an antibody that binds to an h-fat2 polypeptide.

A still further embodiment of the invention is a method for identifying a polypeptide having the ability to control expression of genes implicated in a biological condition selected from the group consisting of malignancy, growth, and differentiation, by providing a host cell capable of expressing an h-fat2 polypeptide, incubating the host cell under conditions that allow expression of the polypeptide, and contacting the host cell or host cell extracts with a labeled antibody that binds to an h-fat2 polypeptide.

Another embodiment of the invention is a method of reducing a malignancy in a population of cells by providing a therapeutic agent comprising an h-fat2 polypeptide or a modulator of an h-fat2 polypeptide, and contacting the population of cells with an effective amount of the therapeutic agent.

Another embodiment of the invention is a pharmaceutical composition having an effective amount of a therapeutic agent comprising an h-fat2 polypeptide or a modulator of a functional portion of a polypeptide having the sequence of SEQ ID NO: 2, and a pharmaceutically acceptable carrier.

Another embodiment of the invention is a method of diagnosis of a malignancy or other biological condition involving hfat-2 expression, expression of hfat-2 variants, underexpression of hfat-2, or lack of hfat-2 expression by providing a polynucleotide probe complementary to at least 12 contiguous nucleotides of an hfat-2 gene sequence, and contacting the probe with mRNA isolated from tissue suspected of overexpressing hfat-2, underexpressing hfat-2, or expressing hfat-2 variants in the process of developing a malignancy or other biological condition.

Another embodiment of the invention is a method of diagnosis of a malignancy or other biological condition implicating hfat-2 expression, expression of hfat-2 variants, underexpression of hfat-2, or lack of hfat-2 expression by providing PCR primers specific for hfat-2 gene sequence or a bDNA probe specific for an hfat-2 gene sequence, contacting the PCR primers or the bDNA probe with hfat-2 DNA or hfat-2 RNA, and detecting mutations in hfat-2 gene sequence by PCR amplification or bDNA probe detection.

Another embodiment of the invention is a method of diagnosis of a malignancy or other biological condition implicating hfat-2 expression, expression of hfat-2 variants, underexpression of hfat-2, or lack of hfat-2 expression by providing an antibody specific for an h-fat2 polypeptide, or an antibody specific to an expressed hfat-2 variant, and detecting qualitative changes in hfat-2 protein expression with the antibody.

The invention also relates to determining the metastic potential of prostate cancer tissue, or PSA-positive cells, by measuring hfat-2 expression in such tissue or cells.

### DETAILED DESCRIPTION OF THE INVENTION

The invention relates to a human fat-2 (hfat-2) polypeptide, comprising a sequence of at least 50 consecutive amino acids from SEQ ID NO: 2, and to a polynucleotide encoding the polypeptide. The gene may relate to the Drosophila fat gene, which is capable of suppressing tumor growth. Expression patterns of hfat-2 in prostate cancer cell lines and tissues, as disclosed herein, show an association between decreased hfat-2 expression and prostate cancer, particularly metastatic prostate cancer.

### Biology of hfat-2

Recessive lethal mutations in the fat locus of Drosophila cause hyperplastic, tumor-like overgrowth of larval imaginal discs, defects in differentiation and morphogenesis, and death during the pupal stage, as described in Mahoney et al., Cell 67:853-869 (1991). The fat locus encodes a member of the cadherin gene superfamily and is a transmembrane protein of over 5000 amino acids, comprising a putative signal sequence, 34 tandem cadherin domains, four epithelial growth factor-like (EGF) repeats, a transmembrane domain, and a novel cytoplasmic domain, also as described in Mahoney et al., Cell 67:853-869 (1991). Mahoney et al. determined that this fly gene is a novel member of the cadherin gene superfamily, functions as a tumor suppressor gene, and is required in the fly for correct morphogenesis.

All members of the cadherin superfamily studied to date are characterized by conserved repeated amino acid sequences (cadherin repeats) in the extracellular domain. A subfamily of the cadherin superfamily includes a group of genes that include the Drosophila fat gene; the encoded proteins have extracellular domains having more than 4 cadherin repeats and an intracellular domain that has a unique sequence. Cadherins mediate calcium-dependent cell-cell adhesion in a wide variety of tissues and are important regulators of morphogenesis, as described in Takeichi, Current Opin. Cell Bio. 5:806-811 (1993), and Geiger and Avalon, Annu. Rev. Cell Biol. 8: 307-332 (1992).

A gene having homology to the Drosophila fat gene was described in Dunne et al, Genomics 30:207-223 (1995). Referred to herein as human fat-1 (hfat-1), the gene was isolated from a T-leukemia cell line.

The present invention relates to a new member of this family, a polynucleotide that comprises the sequences disclosed in SEQ ID NOS: 1 and 3. The putative translation to about 730 amino acids is disclosed in SEQ ID NO: 2. The 3′ sequence of hfat-2 is found in SEQ ID NO: 3.

The fat gene of Drosophila is involved in tumor repression. As described by Gateff, Int. J. Dev. Biol. 38:565-590 (1994), generally the mutation of one recessive gene can precipitate the onset of tumor growth. As disclosed herein, the human hfat-2 gene of the invention is implicated in human cancer, particularly prostate cancer.

Total RNA was extracted from human tissues and cultured cell lines, representing a spectrum from normal epithelial cells through metastatic prostate cell lines and tissue. RNA corresponding to hfat-2 was amplified using PCR and primers specific to the hfat-2 sequence.

hfat-2 RNA in primary epithelial cell culture (PrEC) was strongly positive. A benign prostatic tissue sample (D-803) was moderately positive. In contrast, malignant prostate cancer cell lines were negative, as was a metastatic prostate adenocarcinoma tissue, designated 72-A. The cell lines are as follows: ALVA-31 (Prostate 22:93-108 (1993)); DU145 (Int. J. Cancer 21:274-281 (1978)); JCA-1 (Virology 36:79-84 (1990)); LNCaP (Cancer Res. 43:1809-1818 (1983)); PC-3 (Invest. Virol. 17:16-23 (1979)); PPC-1 (Int. J. Cancer 44:898-903 (1989)); TSU-Pr1 (J. Virol. 137:1304-1306 (1987)).

Although the invention is not limited by any theories of mechanism, mutations in a tumor suppressor gene can result in a loss of tumor suppressor function which in turn can result in tumor cell growth in the animal. In the specific case of Drosophila fat, loss of function results in induction of malignant neoplastic transformation of larval plasmatocytes. In a mammalian cancer context, loss of functional hfat-2 may, for example, cause the organism to lose a differentiated state in a population of cells, or may be responsible for expression of genes that cause this loss; in addition or alternatively, hfat-2 mutations may be responsible for a population of cells, such as stem cells, never gaining a differentiated state as occurs in a normal condition. Loss of function of hfat-2 in a cancer context may also be responsible, for example, for stem cells or other cells to acquire a new developmental fate.

Expression of hfat-2 in a therapeutic context, for example in a gene therapy protocol, may promote normal growth or normal development in biological conditions where aberrant expression or loss of function of hfat-2 is implicated, having detrimental effects on the growth or development of an organism.

### Construction of Polypeptides of the Invention and Variants Thereof

Polypeptides of the invention include those encoded by a polynucleotide comprising part or all of SEQ ID NO: 1 and/or 3. Polypeptides of the invention also can be encoded by nucleic acids that, by virtue of the degeneracy of the genetic code, are not identical in sequence to the disclosed hfat-2 sequence. Thus, the invention includes within its scope nucleic acids comprising polynucleotides encoding a protein or polypeptide expressed by a polynucleotide comprising the sequence of SEQ ID NO: 1 and/or 3. Also within the scope of the invention are variants; variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. For example, substitutions between the following groups are conservative: Gly/Ala, Val/Ile/Leu, Asp/Glu, Lys/Arg, Asn/Gln, Ser/Cys, Thr, and Phe/Trp/Tyr.

The variant may be designed so as to retain biological activity of a particular region of the protein. In a non-limiting example, Osawa et al., Biochemistry and Molecular International 34:1003-1009 (1994), discusses homologous proteins having amino acids that fall within “homologous residue groups.” Homologous residues are judged according to the following groups (using single letter amino acid designations): STAG; ILVMF; HRK; DEQN; and FYW. For example, and S, a T, an A or a G can be in a position and the function generally is retained.

Cysteine-depleted muteins are considered variants within the scope of the invention. These variants can be constructed according to methods disclosed in U.S. Pat. No. 4,959,314, “Cysteine-Depleted Muteins of Biologically Active Proteins.” The patent discloses how to substitute other amino acids for cysteines, and how to determine biological activity and effect of the substitution. Such methods are suitable for proteins according to this invention that have cysteine residues suitable for such substitutions, for example to eliminate disulfide bond formation.

The protein variants described herein are encoded by polynucleotides that are within the scope of the invention. The genetic code can be used to select the appropriate codons to construct the corresponding variants.

Any naturally occurring variants of the nucleotide sequences which encode polypeptide variants thereof are within the scope of this invention (with the exception of hfat-1). Allelic variants of subgenomic polynucleotides of the invention can occur and can be identified by hybridization of putative allelic variants with nucleotide sequences disclosed herein under stringent conditions. For example, by using the following wash conditions—2×SCC, 0.1% SDS, room temperature twice, 30 minutes each; then 2×SCC, 0.1% SDS, 50° C. once, 30 minutes; then 2×SCC, room temperature twice, 10 minutes each—allelic variants of the polynucleotides of the invention can be identified which contain at most about 25-30% base pair mismatches. More preferably, allelic variants contain 15-25% base pair mismatches, even more preferably 5-15%, or 2-5%, or 1-2% base pair mismatches. A single base-pair mismatch is also within the scope of an allelic variation.

For the purposes of this invention, a preferred method of calculating percent identity is the Smith-Waterman algorithm, using the following. Global DNA sequence identity must be greater than 65% as determined by the Smith-Waterman homology search algorithm as implemented in MPSRCH program (Oxford Molecular) using an affine gap search with the following search parameters: gap open penalty, 12; and gap extension penalty, 1.

Amplification by the polymerase chain reaction (PCR) can be used to obtain the polynucleotides of the invention, using either genomic DNA or cDNA as a template. The polynucleotides of the invention may also be obtained using reverse transcriptase and mRNA molecules that are complementary to the minus strand of a double-stranded sequence wherein said double-stranded sequence is selected from the group of polynucleotides comprising a sequence as shown in SEQ ID NOS: 1 and 3. Using the polynucleotide sequences disclosed herein, subgenomic polynucleotide molecules of the invention can also be made using known techniques of synthetic chemistry.

Probes specific to the polynucleotides of the invention may be generated using the polynucleotide sequences disclosed in SEQ ID NOS: 1 and 3. The probes are preferably at least 12, 14, 16, 18, 20, 22, 24, or 25 nucleotides in length and can be less than 2, 1, 0.5, 0.1, or 0.05 kb in length. Preferred probes are capable of uniquely hybridizing to hfat-2-encoding polynucleotides. The probes can be synthesized chemically or can be generated from longer polynucleotides using restriction enzymes. The probes can be labeled, for example, with a radioactive, biotinylated, or fluorescent tag.

### Use of the hfat-2 gene, Related Polynucleotides, and Encoded Polypeptides to Raise Antibodies

Expression products of a polynucleotide, the corresponding mRNA or cDNA, or the corresponding complete gene are prepared and used for raising antibodies for experimental, diagnostic, and therapeutic purposes. These antibodies are specific to an epitope on the hfat-2-encoded polypeptide, and can precipitate or bind to the corresponding native protein in a cell or tissue preparation or in a cell-free extract of an in vitro expression system.

Immunogens for raising antibodies can be prepared by mixing a polypeptide encoded by SEQ ID NO: 1, SEQ ID NO: 3, or the corresponding complete hfat-2 gene, with adjuvants. Alternatively, polypeptides are made as fusion proteins to larger immunogenic proteins. Polypeptides are also covalently linked to other larger immunogenic proteins, such as keyhole limpet hemocyanin. Immunogens are typically administered intradermally, subcutaneously, or intramuscularly. Immunogens are administered to experimental animals such as rabbits, sheep, and mice, to generate antibodies. Optionally, the animal spleen cells are isolated and fused with myeloma cells to form hybridomas that secrete monoclonal antibodies. Such methods are well known in the art. According to another method known in the art, the polynucleotide is administered directly, such as by intramuscular injection, and expressed in vivo. The expressed protein generates a variety of protein-specific immune responses, including production of antibodies, comparable to administration of the protein.

Preparations of polyclonal and monoclonal antibodies specific for the encoded proteins and polypeptides are made using standard methods known in the art. The antibodies specifically bind to epitopes present in the polypeptides encoded by polynucleotides disclosed in the Sequence Listing. Typically, at least 6, 8, 10, or 12 contiguous amino acids are required to form an epitope. However, epitopes that involve non-contiguous amino acids may require more, for example at least 15, 25, or 50 amino acids.

Antibodies that specifically bind to human hfat-2-encoded polypeptides should provide a detection signal at least 5-, 10-, or 20-fold higher than a detection signal provided with other proteins when used in Western blots or other immunochemical assays. Preferably, antibodies that specifically bind hfat-2-encoded polypeptides do not detect other proteins in immunochemical assays and can immunoprecipitate hfat-2-encoded proteins from solution.

In addition to the antibodies discussed above, genetically engineered antibody derivatives are made, such as single chain antibodies.

### Use of hfat-2 Gene and Related Polynucleotides to Construct Arrays for Diagnostics

To create arrays, polynucleotide probes are spotted onto a substrate in a two-dimensional matrix or array. Samples of polynucleotides can be labeled and then hybridized to the probes. Double stranded polynucleotides, comprising the labeled sample polynucleotides bound to probe polynucleotides, can be detected once the unbound portion of the sample is washed away.

The probe polynucleotides can be spotted on substrates including glass, nitrocellulose, etc. The probes can be bound to the substrate by either covalent bonds or by non-specific interactions, such as hydrophobic interactions. The sample polynucleotides can be labeled using radioactive labels, fluorophors, etc.

Techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. No. 5,593,839; U.S. Pat. No. 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734.

Further, arrays can be used to examine differential expression of genes and can be used to determine gene function. For example, arrays of the instant polynucleotide sequences can be used to detect expression of hfat-2 in normal cells and cancer cells.

### Ribozymes

Trans-cleaving catalytic RNAs (ribozymes) are RNA molecules possessing endoribonuclease activity. Ribozymes are specifically designed for a particular target, and the target message must contain a specific nucleotide sequence. They are engineered to cleave any RNA species site-specifically in the background of cellular RNA. The cleavage event renders the mRNA unstable and prevents protein expression. Importantly, ribozymes can be used to inhibit expression of a gene of unknown function for the purpose of determining its function in an in vitro or in vivo context, by detecting the phenotypic effect.

One commonly used ribozyme motif is the hammerhead, for which the substrate sequence requirements are minimal. Design of the hammerhead ribozyme is disclosed in Usman et al., Current Opin. Struct. Biol. 6:527-533 (1996). Usman also discusses the therapeutic uses of ribozymes. Ribozymes can also be prepared and used as described in Long et al., FASEB J. 7:25 (1993); Symons, Ann. Rev. Biochem. 61:641 (1992); Perrotta et al., Biochem. 31:16-17 (1992); Ojwang et al., Proc. Natl. Acad. Sci. (USA) 89:10802-10806 (1992); and U.S. Pat. No. 5,254,678. Ribozyme cleavage of HIV-I RNA is described in U.S. Pat. No. 5,144,019; methods of cleaving RNA using ribozymes is described in U.S. Pat. No. 5,116,742; and methods for increasing the specificity of ribozymes are described in U.S. Pat. No. 5,225,337 and Koizumi et al., Nucleic Acid Res. 17:7059-7071 (1989). Preparation and use of ribozyme fragments in a hammerhead structure are also described by Koizumi et al., Nucleic Acids Res. 17:7059-7071 (1989). Preparation and use of ribozyme fragments in a hairpin structure are described by Chowrira and Burke, Nucleic Acids Res. 20:2835 (1992). Ribozymes can also be made by rolling transcription as described in Daubendiek and Kool, Nat. Biotechnol. 15(3):273-277 (1997).

The hybridizing region of the ribozyme may be modified or may be prepared as a branched structure as described in Horn and Urdea, Nucleic Acids Res. 17:6959-67 (1989). The basic structure of the ribozymes may also be chemically altered in ways familiar to those skilled in the art, and chemically synthesized ribozymes can be administered as synthetic oligonucleotide derivatives modified by monomeric units. In a therapeutic context, liposome mediated delivery of ribozymes improves cellular uptake, as described in Birikh et al., Eur. J. Biochern. 245:1-16 (1997). Using the sequences of the invention and methods known in the art, ribozymes are designed to specifically bind and cut the corresponding mRNA species.

### Antisense

Antisense nucleic acids are designed to specifically bind to RNA, resulting in the formation of RNA-DNA or RNA-RNA hybrids, with an arrest of DNA replication, reverse transcription or messenger RNA translation. Antisense polynucleotides based on a selected polynucleotide sequence of the invention can interfere with expression of the corresponding gene. Antisense polynucleotides are typically generated within the cell by expression from antisense constructs that contain the antisense strand as the transcribed strand. Antisense encoded polynucleotides will bind and/or interfere with the translation of related mRNA. The expression products of control cells and cells treated with the antisense construct are compared to detect the protein products related to the gene corresponding to the antisense construct. The proteins are isolated and identified using routine biochemical methods.

### Use of hfat-2, Related Polynucleotides. and Encoded Polypeptides to Screen for Peptide Analogs and Antagonists

Polypeptides encoded by the polynucleotides of the invention can be used to screen peptide libraries to identify binding partners from among the encoded polypeptides.

A library of peptides may be synthesized following the methods disclosed in U.S. Pat. No. 5,010,175, and in PCT WO 91/17823. As described below in brief, one prepares a mixture of peptides, which is then screened to identify peptides exhibiting the desired activity. In the '175 method, a suitable peptide synthesis support (e.g., a resin) is coupled to a mixture of appropriately protected, activated amino acids. The concentration of each amino acid in the reaction mixture is balanced or adjusted in inverse proportion to its coupling reaction rate so that the product is an equimolar mixture of amino acids coupled to the starting resin. The bound amino acids are then deprotected, and reacted with another balanced amino acid mixture to form an equimolar mixture of all possible dipeptides. This process is repeated until a mixture of peptides of the desired length (e.g., hexamers) is formed. One need not include all amino acids in each step: one may include only one or two amino acids in some steps (e.g., where it is known that a particular amino acid is essential in a given position), thus reducing the complexity of the mixture. After the synthesis of the peptide library is completed, the mixture of peptides is screened for binding to the selected polypeptide. The peptides are then tested for their ability to inhibit or enhance activity. Peptides exhibiting the desired activity are then isolated and sequenced.

Peptide agonists or antagonists are screened using any available method, such as signal transduction, antibody binding, receptor binding, mitogenic assays, chemotaxis assays, etc. The methods described herein are presently preferred. The assay conditions ideally should resemble the conditions under which the native activity is exhibited in vivo, that is, under physiologic pH, temperature, and ionic strength.

Suitable agonists or antagonists will exhibit strong inhibition or enhancement of the native activity at concentrations that do not cause toxic side effects in the subject. Agonists or antagonists that compete for binding to the native polypeptide may require concentrations equal to or greater than the native concentration, while inhibitors capable of binding irreversibly to the polypeptide may be added in concentrations on the order of the native concentration.

The end results of such screening and experimentation will be at least one peptide agonist or antagonist of hfat-2. Such agonists and antagonists can be used to modulate, enhance, or inhibit function in cells to which the hfat-2 is native, or in cells that possess hfat-2 as a result of genetic engineering.

### Pharmaceutical Compositions and Therapeutic Uses

Pharmaceutical compositions can comprise polypeptides, antibodies, or polynucleotides of the claimed invention. The pharmaceutical compositions will comprise a therapeutically effective amount of either polypeptides, antibodies, or polynucleotides.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgment of the clinician.

For purposes of the present invention, an effective dose will be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

### Delivery Methods

Once formulated, the polynucleotide compositions of the invention can be (1) administered directly to the subject; (2) delivered ex vivo, to cells derived from the subject; or (3) delivered in vitro for expression of recombinant proteins.

Direct delivery of the compositions will generally be accomplished by injection, either subcutaneously, intraperitoneally, intravenously or intramuscularly, or delivered to the interstitial space of a tissue. The compositions can also be administered into a tumor or lesion. Other modes of administration include oral and pulmonary administration, suppositories, and transdermal applications, needles, and gene guns or hyposprays. Dosage treatment may be a single dose schedule or a multiple dose schedule.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoetic, lymph cells, macrophages, dendritic cells, or tumor cells.

Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Preparation of antisense polypeptides is discussed above. Neoplasias that are treated with the antisense composition include, but are not limited to, prostate cancers, cervical cancers, melanomas, colorectal adenocarcinomas, Wilms' tumor, retinoblastoma, sarcomas, myosarcomas, lung carcinomas, leukemias, such as chronic myelogenous leukemia, promyelocytic leukemia, monocytic leukemia, and myeloid leukemia, and lymphomas, such as histiocytic lymphoma. Proliferative disorders that are treated with the therapeutic composition include disorders such as anhydric hereditary ectodermal dysplasia, congenital alveolar dysplasia, epithelial dysplasia of the cervix, fibrous dysplasia of bone, and mammary dysplasia. Hyperplasias, for example, endometrial, adrenal, breast, prostate, or thyroid hyperplasias or pseudoepitheliomatous hyperplasia of the skin, are treated with antisense therapeutic compositions. Even in disorders in which mutations in the corresponding gene are not implicated, downregulation or inhibition of related gene expression can have therapeutic application.

For example, decreasing related gene expression can help to suppress tumors in which enhanced expression of the gene is implicated.

Both the dose of the antisense composition and the means of administration are determined based on the specific qualities of the therapeutic composition, the condition, age, and weight of the patient, the progression of the disease, and other relevant factors. Administration of the therapeutic antisense agents of the invention includes local or systemic administration, including injection, oral administration, particle gun or catheterized administration, and topical administration. Preferably, the therapeutic antisense composition contains an expression construct comprising a promoter and a polynucleotide segment of at least 12, 22, 25, 30, or 35 contiguous nucleotides of the antisense strand. Within the expression construct, the polynucleotide segment is located downstream from the promoter, and transcription of the polynucleotide segment initiates at the promoter.

Various methods are used to administer the therapeutic composition directly to a specific site in the body. For example, a small metastatic lesion is located and the therapeutic composition injected several times in several different locations within the body of tumor. Alternatively, arteries which serve a tumor are identified, and the therapeutic composition injected into such an artery, in order to deliver the composition directly into the tumor. A tumor that has a necrotic center is aspirated and the composition injected directly into the now empty center of the tumor. The antisense composition is directly administered to the surface of the tumor, for example, by topical application of the composition. X-ray imaging is used to assist in certain of the above delivery methods.

Receptor-mediated targeted delivery of therapeutic compositions containing an antisense polynucleotide, subgenomic polynucleotides, or antibodies to specific tissues is also used. Receptor-mediated DNA delivery techniques are described in, for example, Findeis et al., Trends in Biotechnol. (1993) 11:202-205; Chiou et al., (1994) Gene Therapeutics: Methods And Applications Of Direct Gene Transfer (J. A. Wolff, ed.); Wu & Wu, J. Biol. Chem. (1988) 263:621-24; Wu et al., J. Biol. Chem. (1994) 269:542-46; Zenke et al., Proc. Natl. Acad. Sci. (USA) (1990) 87:3655-59; Wu et al., J. Biol. Chem. (1991) 266:338-42. Preferably, receptor-mediated targeted delivery of therapeutic compositions containing antibodies of the invention is used to deliver the antibodies to specific tissue.

### Gene Therapy

The therapeutic polynucleotides and polypeptides of the present invention may be utilized in gene delivery vehicles. The gene delivery vehicle may be of viral or non-viral origin (see generally, Jolly, Cancer Gene Therapy 1:51-64 (1994); Kimura, Human Gene Therpay 5:845-852 (1994); Connelly, Human Gene Therapy 1:185-193 (1995); and Kaplitt, Nature Genetics 6:148-153 (1994)). Gene therapy vehicles for delivery of constructs including a coding sequence of a therapeutic of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0 415 731; WO 90/07936; WO 94/03622; WO 93/25698; WO 93/25234; U.S. Pat. No. 5,219,740; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127; GB Patent No. 2,200,651; and EP 0 345 242. Preferred recombinant retroviruses include those described in WO 91/02805.

Packaging cell lines suitable for use with the above-described retroviral vector constructs may be readily prepared (see PCT publications WO 95/30763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

The present invention also employs alphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. 63:3822-3828 (1989); Mendelson et al., Virol. 166:154-165 (1988); and Flotte et al., PNAS 90:10613-10617 (1993).

Representative examples of adenoviral vectors include those described by Berkner, Biotechniques 6:616-627 (1988); Rosenfeld et al., Science 252:431-434 (1991); WO 93/19191; Kolls et al., PNAS 91:215-219 (1994); Kass-Eisler et al., PNAS 90:11498-11502 (1993); Guzman et al., Circulation 88:2838-2848 (1993); Guzman et al., Cir. Res. 73:1202-1207 (1993); Zabner et al., Cell 75:207-216 (1993); Li et al., Hum. Gene Ther. 4:403-409 (1993); Cailaud et al., Eur. J. Neurosci. 5:1287-1291 (1993); Vincent et al., Nat. Genet. 5:130-134 (1993); Jaffe et al., Nat. Genet. 1:372-378 (1992); and Levrero et al., Gene 101:195-202 (1991). Exemplary adenoviral gene therapy vectors employable in this invention also include those described in WO 94/12649, WO 93/03769; WO 93/19191; WO 94/28938; WO 95/11984 and WO 95/00655. Administration of DNA linked to killed adenovirus as described in Curiel, Hum. Gene Ther. 3:147-154 (1992), may be employed.

Other gene delivery vehicles and methods may be employed, including polycationic condensed DNA linked or unlinked to killed adenovirus alone, for example Curiel, Hum. Gene Ther. 3:147-154 (1992); ligand linked DNA, for example see Wu, J. Biol. Chem. 264:16985-16987 (1989); eukaryotic cell delivery vehicles cells, for example see U.S. Ser. No. 08/240,030, filed May 9, 1994, and U.S. Ser. No. 08/404,796; deposition of photopolymerized hydrogel materials; hand-held gene transfer particle gun, as described in U.S. Pat. No. 5,149,655; ionizing radiation as described in U.S. Pat. No. 5,206,152 and in WO92/11033; nucleic charge neutralization or fuision with cell membranes. Additional approaches are described in Philip, Mol. Cell Biol. 14:2411-2418 (1994), and in Woffendin, Proc. Natl. Acad. Sci. 91:1581-1585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Nos. WO 95/13796, WO 94/23697, and WO 91/14445, and EP No. 0 524 968.

Further non-viral delivery suitable for use includes mechanical delivery systems such as the approach described in Woffendin et al., Proc. Natl. Acad. Sci. USA 91(24):11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT No. WO 92/11033.

### EXAMPLES

### Example 1

### Expression of HFAT-2

Poly A+RNA was isolated from a normal adult cerebellum. The mRNA was electrophoretically fractionated and transferred to a nylon filter. The mRNA on the filter was immobilized by UV crosslinking. A labeled probe was prepared from the sequence of SEQ ID NO: 1, labeled with p³² radionucleotide, and used in a hybridization reaction with the RNA on the filter under stringent conditions.

The filter was allowed to hybridize to the probe, and the unbound probe was washed from the filter. The hybridization was conducted using standard techniques for Northern hybridizations as described in Sambrook et al. (1989), MOLECULAR CLONING: A LABORATORY MANUAL, 2nd ed. (Cold Spring Harbor Press, Cold Spring Harbor, N.Y.).

Exposure of the filter to X-ray film showed pronounced bands in the cerebellum-derived tissue of the expected size of the h-fat2 transcript. Beta actin was used as a control to normalize expression levels in the cell lines.

### Example 2

### Lack of Expression of HFAT-2 in Metastatic Prostate Cancer Cell Lines and Tissue

Total RNA was extracted from the cultured cells and human tissues indicated below as follows (Chomczynski and Sacchi, Anal. Biochem. 162(1):156-159 (1987)). Cells were lysed in guanidinium isothiocyanate, then phenol:chloroform extracted at pH 4.0 followed by isopropanol precipitation of the aqueous layer. The pellet was re-dissolved in guanidinium isothiocyanate and re-precipitated with isopropanol. That pellet was washed 2× with 75% ethanol, then dried briefly before resuspending in DEPC-treated water. After quantification by absorbance ratio (260/280) and visualization on an agarose gel, reverse transcription using Promega MMLV reverse transcriptase (50 U/500 ng RNA) and random hexamers (2.5 μM) was carried out following Dnase I treatment.

cDNA resulting from 250 ng of RNA was used as template in a 25 μl PCR reaction. PCR conditions were as follows: hfat-2-specific primers (0.25 μM each), dNTP's −200 μM, Promega Taq 0.025 U/μl, magnesium chloride 1.5 mM in manufacturer's 1× reaction buffer. Cycling was as follows: 94° C. for 4 minutes; 94° C. for 1 minute/68° C. for 1 minute for 25 or 35 cycles; extension 72° C. for 5 minutes. Products were separated on 2% NuSieve agarose in 0.5×TBE, and visualized with ethidium bromide.

### RNA Sources

The following established cell lines were used:

| | |
|---|---|
| ALVA-31 | (Prostate 22:93-108 (1993)) |
| DU145 | (Int. J. Cancer 21:274-281 (1978)) |
| JCA-1 | (Urology 36:79-84 (1990)) |
| LNCaP | (Cancer Res 43:1809-1818 (1983)) |
| PC-3 | (Invest. Urol. 17:16-23 (1979)) |
| PPC-1 | (Int. J. Cancer 44:898-903 (1989)) |
| TSU-Pr1 | (J. Urol. 137:1304-1306 (1987)) |

These cell lines were authenticated by karyotype.

Primary prostate epithelial cell cultures: PrEC (Clonetics).

The following primary tissues were used:

D803 Histologically normal prostate from organ donor

72-A Metastatic prostatic adenocarcinoma to pelvic lymph node

### Results

At 25 cycles, the benign tissue (D803) was moderately positive and the primary epithelial cell culture was strongly positive. All of the malignant cell lines and the metastatic carcinoma specimen (72-A) were essentially negative.

Twenty-five cycles are normally performed to detect differences in expression. To amplify and detect very low levels of expression, 35 cycles can be performed. At 35 cycles, using a hfat-2-specific primer set, the following differences between the cell lines were observed (Tables 1 and 2).

TABLE 1

| Cell Line | Relative Expression |
|---|---|
| ALVA-31 | 3+ |
| DU-145 | 1+ |
| JCA-1 | 1+ |
| LNCaP | 3+ |
| PPC-1 | 2+ |
| TSU-Pr1 | 2+ |

Under these conditions PrEc exhibits an intense level (10+).

TABLE 2

| Cell Line | Relative Expression |
|---|---|
| ALVA-3 1 | 4+ |
| DU-145 | 1+ |
| JCA-1 | 3+ |
| LNCaP | 4+ |
| PC-3 | 2+ |
| PPC-1 | 2+ |
| TSU-Pr1 | 2+ |

PrEc was also a 4+, although under the 35 cycle regimen expression levels in cells previously shown as positive can be distorted due to saturation. Thus, the 35 cycle comparison is most valid for comparing highly amplified expression in cell lines which, at 25 cycles, exhibited undetectable levels.

### Example 3

### Diagnostic Uses of HFAT-2

In a non-limiting example, hfat-2 expression by PSA-positive (Prostate Specific Antigen-Positive) cells in the blood, or in a prostate tissue biopsy, can be measured using methods described above. Higher hfat-2 expression can correlate with a lower extent or risk of metastasis. Absence or lower levels of hfat-2 expression, in contrast, can correlate with a greater risk of metastasis.

Although the foregoing invention has been described in some detail by way of illustration and examples, one of skill in the art will recognize that certain changes and modifications may be practiced within the scope of the appended claims.

What is claimed is:

1. An isolated polynucleotide selected from the group consisting of:
(a) a polynucleotide comprising the nucleotide sequence of SEQ ID NO: 1;
(b) a polynucleotide comprising the nucleotide sequence of SEQ ID NO: 1 and SEQ ID NO: 3;
(c) mRNA obtained by hybridizing human cytoplasmic RNA with a probe based on SEQ ID NO: 1 or SEQ ID NO: 3, wherein said probe is of a length adequate to uniquely identify said gene;
(d) a polynucleotide encoding a protein comprising the amino acid sequence of SEQ ID NO: 2;
(e) a polynucleotide encoding a variant of the amino acid sequence of SEQ ID NO: 2, wherein said variant has the biological activity of hfat-2 protein; and
(f) a polynucleotide that is an allelic variant of a polynucleotide of (a)-(e) above, wherein said polynucleotide does not encode human fat-1 protein.

2. A composition comprising a protein, wherein said protein comprises an amino acid sequence selected from the group consisting of:
(a) the amino acid sequence of SEQ ID NO: 2; and
(b) a biologically active fragment of the amino acid sequence of SEQ ID NO: 2.

3. The polynucleotide of claim 1 comprising the nucleotide sequence of SEQ ID NO: 1.

4. A vector comprising a polynucleotide of claim 1, wherein said polynucleotide is operably linked to an expression control sequence.

5. A host cell transformed with the vector of claim 4.

6. The host cell of claim 5, wherein said host cell is a mammalian cell.

7. An animal comprising a heterologous host cell, wherein said host cell comprises the nucleotide sequence of claim 1.

8. An animal comprising a heterologous host cell, wherein said host cell comprises the polypeptide of claim 2.

9. A method for producing a protein, said method comprising:
(a) growing a culture of the host cell of claim 5 in a suitable culture medium; and
(b) purifying the protein from the culture medium.

10. A method of inhibiting the growth of cancer cells, said method comprising exposing said cancer cells to a polypeptide encoded by at least one of SEQ ID NO: 1 and SEQ ID NO: 3.

11. The method of claim 10, wherein said cancer cells are prostate cancer cells.

12. A method of determining the metastatic potential of a prostate cancer cell, said method comprising:
(a) obtaining mRNA from a prostate cancer tissue or from prostate-specific antigen-positive cells;
(b) exposing said mRNA to a polynucleotide probe comprising at least 15 contiguous nucleotides of SEQ ID NO: 1 or SEQ ID NO: 3 under stringent hybridization conditions to allow duplex formation between said mRNA and said probe; and
(c) detecting duplexes, wherein presence of duplexes is correlated with low metastatic potential.

13. The method of claim 12, wherein said mRNA of step (a) is amplified, and said amplified polynucleotide is exposed to said probe of step (b).
